(19) United States
(12) Patent Application Publication
(10) Pub. No.: US 2025/0136915 A1
(43) Pub. Date: May 1, 2025

(54) AUTOMATED NEEDLE-BASED SAMPLE COLLECTOR FOR BIOREACTORS

(71) Applicant: GEORGIA TECH RESEARCH CORPORATION, Atlanta, GA (US)

(72) Inventors: Walker Thomas BYRNES, Atlanta, GA (US); Stephen BALAKIRSKY, Atlanta, GA (US); Annie BOWLES-WELCH, Atlanta, GA (US); Bharat KANWAR, Atlanta, GA (US); Krishnendu ROY, Marietta, GA (US); Bryan WANG, Atlanta, GA (US); Carolyn YEAGO, Atlanta, GA (US)

(21) Appl. No.: 18/836,216

(22) PCT Filed: Feb. 7, 2023
(86) PCT No.: PCT/US2023/012491

§ 371 Date: Aug. 6, 2024

Related U.S. Application Data
(60) Provisional application No. 63/307,570, filed on Feb. 7, 2022.

(51) Int. Cl. C12M 1/26; C12M 1/36

(52) U.S. Cl. CPC C12M 33/04; C12M 41/48

(57) ABSTRACT

An automated sampling platform includes a gantry-style 2D or 3D actuator equipped with a syringe pump end effector configured to extract samples from a rubber septum placed in-line within a tubing-based system and to deposit the extracted samples into sealed cryovials for storage. This device can be employed for use in cell culture bioreactors, such as the Terumo Quantum, which include a waste line.

### CROSS REFERENCE TO RELATED APPLICATIONS

This application claims priority benefit of U.S. Provisional Patent Application Ser. No. 63/307,570, filed Feb. 7, 2022, pending, which application is hereby incorporated by this reference in its entirety for all purposes as if fully set forth herein.

### ACKNOWLEDGEMENTS

This invention was made with government support under award no. R01FD006598-03 awarded by the Food and Drug Administration. This invention was made with government support under contract no. 1648035 awarded by the National Science Foundation (NSF). The government has certain rights in the invention.

### BACKGROUND

### Field

Embodiments of the present invention relate an automated sampling platform with the capability to draw samples at-line without human interference, maintaining the sterility of the system. to a syringe-based sample collector, specifically an automated needle-based sample collector for bioreactors.

### Background

Cell therapy is a developing field with different cell therapy products showing promising clinical results for immune regulation, inflammation, cancer, and other indications. These cell therapy products require large-scale bioreactors, such as the Quantum by Terumo BCT, to produce cell therapies in clinically relevant quantities. However, they rely on manual procedures and skilled operators to take samples for media/cell analysis and update process parameters during the expansion.

The large variety in modern bioreactor design has led to highly specific or impractical options for automated sample collection and storage devices. As a result, researchers in cell manufacturing frequently resort to manual sampling, which is time-consuming and exposes the bioreactor system to outside contaminants. Automated sampling devices for use with bioreactors have been developed and are readily available as commercial products. Multiple commercial devices exist to collect and transfer samples, both into storage vials (1) or downstream analyzers (2). Low-cost open-source bioreactor samplers have also been released open-source so that labs can construct their own devices (3). However, all of these products utilize tubing lines and peristaltic pumps for sample collection and distribution. Conventional automated sampling interfaces either use automatic pipetting or require drawing media from tubing volumes.

Present in all of these tubing-based systems in an unavoidable dead volume, sample media required to fill the line of tubing before the sample vessel is reached, which is typically flushed to waste. Drawing from tubing volumes requires large volumes of media from the reactor to take even small samples with the potential of introducing contamination and undue process disturbance by returning the un-sampled liquid to the dead space. Additionally, repeated use of the same tubing lines introduces a risk of cross-contamination between samples, reducing sample integrity and allowing uncertainty in the validity of assays performed on samples. Moreover, automatic pipetting is limited by the sterility requirements of the bioreactor to maintain a closed system,

### BRIEF SUMMARY OF THE DISCLOSURE

Accordingly, the present invention is directed to an automated needle-based sample collector for bioreactors that obviates one or more of the problems due to the limitations and disadvantages of the related art.

In accordance with the purpose(s) of this invention, as embodied and broadly described herein, this invention, in one aspect, relates to an automated sampling system including a syringe sampling assembly; and a sampling platform comprising: a mounting structure; at least one sample storage rack and at least one corresponding sample container; a movable carriage assembly fixably coupled to the mounting structure, the movable carriage assembly comprising a movable carriage configured to carry at least one syringe sampling assembly; a sampling port mount fixably coupled to, or relative to, the mounting structure, the sampling port mount having one or more samples of interest (static or flowing); the syringe sampling assembly coupled to the mounting structure, the syringe sampling assembly having one or more actuated assemblies configured, and directed via a controller, to (i) attachably engage a syringe from a set of syringes, (ii) position the syringe at the sampling port mount, (iii) actuate the syringe to draw a sample from the one or more samples of interest, (iv) position the syringe at the corresponding sample container, (v) actuate the syringe to expel a portion of the sample into the corresponding sample container, (vi) disengage the syringe, and (vii) attachably engage another syringe from a set of syringes for drawing a another sample from the one or more samples of interest different from the previously drawn sample.

In another aspect, an automated sampling system is provided and includes a sampling platform comprising a mounting structure; a movable carriage; a sampling port mount; and a sample storage rack; and a syringe pump assembly removably coupled to the movable carriage.

In yet another aspect, a method of autosampling biosamples is provided and includes using the automated sampling system using systems as described herein, the method comprising: moving the syringe via the moveable carriage to the sampling port mount, inserting a needle of the syringe into a sampling port held in place by the sampling port mount, aspirating a sample, extracting the needle from the sampling port, moving the syringe via the moveable carriage to a vial in the sample storage rack, ejecting the sample into the vial, and returning the needle to the sampling port or to a storage vial.

In yet another aspect, a non-transitory computer-readable medium is provided having instructions stored thereon, wherein execution of the instructions by the processor, causes the processor to perform any one of the methods described herein.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

The herein-described invention allows for the fully automated sampling of media and/or media containing cells at variable sampling frequencies and volumes with no sample cross-contamination, waste or dead volume. Furthermore, in as aspect the samples are able to be automatically frozen for future use.

This represents an improvement over manual approaches in that no skilled technician is required to be present when a sample is drawn. Advantages over other commercially available automated sampling devices include no media waste, no chance of cross-contamination, and a significantly lower possibility of cross-contamination between samples than in other sampling systems.

An advantage of the present invention is to provide for convenient, variable-size samples of media and/or media-containing cells with a very low chance of system contamination. This is performed without human interaction, according to a preset sampling schedule.

Further embodiments, features, and advantages of the automated needle-based sample collector as well as the structure and operation of the various embodiments of the automated needle-based sample collector are described in detail below with reference to the accompanying drawings.

It is to be understood that both the foregoing general description and the following detailed description are exemplary and explanatory only and are not restrictive of the invention as claimed.

### BRIEF DESCRIPTION OF THE DRAWINGS

The accompanying figures, which are incorporated herein and form part of the specification, illustrate an automated needle-based sample collector for bioreactors. Together with the description, the figures further serve to explain the principles of the automated needle-based sample collector for bioreactors described herein and thereby enable a person skilled in the pertinent art to make and use the automated needle-based sample collector for bioreactors.

FIG. 1 shows an example automated sampling platform in accordance with an illustrative embodiment.

FIG. 2 shows top, front, and side views of the system of FIG. 1.

FIG. 3 shows a detailed diagram of the syringe tower and set of modules of FIG. 1.

FIG. 4 shows various components of the automated sampling platform of FIG. 1.

FIG. 5 is a software architecture diagram for software according to the principles described herein.

FIG. 6 illustrates a graphical user interface for scheduling software according to the principles described herein.

FIG. 7 illustrates an example automated sampling platform with temperature-controlled storage.

FIG. 8 illustrates an example freezer storage sub-subsystem for use in conjunction with the example automated sampling platform of FIG. 7.

FIG. 9 illustrates an example gantry system for use with the example automated sampling platform of FIG. 7.

FIG. 10 illustrates an example syringe holder for use according to the principles described herein.

FIG. 11 illustrates a syringe and needle storage box for use according to principles described herein.

FIG. 12 illustrates the snap fit assembly of a syringe into a syringe actuator according to the principles described herein.

FIG. 13 illustrates mechanical compliance provided into an example syringe holder according to the principles described herein.

FIGS. 14A and 14B illustrate a syringe cartridge and a needle uncapping device with respect to an automated sampling system as described herein.

FIG. 15 shows needle cap removal for syringes in an example syringe holder according to the principles described herein.

### DETAILED DESCRIPTION

Reference will now be made in detail to embodiments of the automated needle-based sample collector for bioreactors with reference to the accompanying figures. The same reference numbers in different drawings may identify the same or similar elements.

It will be apparent to those skilled in the art that various modifications and variations can be made in the present invention without departing from the spirit or scope of the invention. Thus, it is intended that the present invention cover the modifications and variations of this invention provided they come within the scope of the appended claims and their equivalents.

Automated sampling systems for bioreactors have been developed previously, but none with this particular set of characteristics. Most existing sampling systems use lines of tubing to transfer samples to their storage locations, which can lead to cross-contamination between samples.

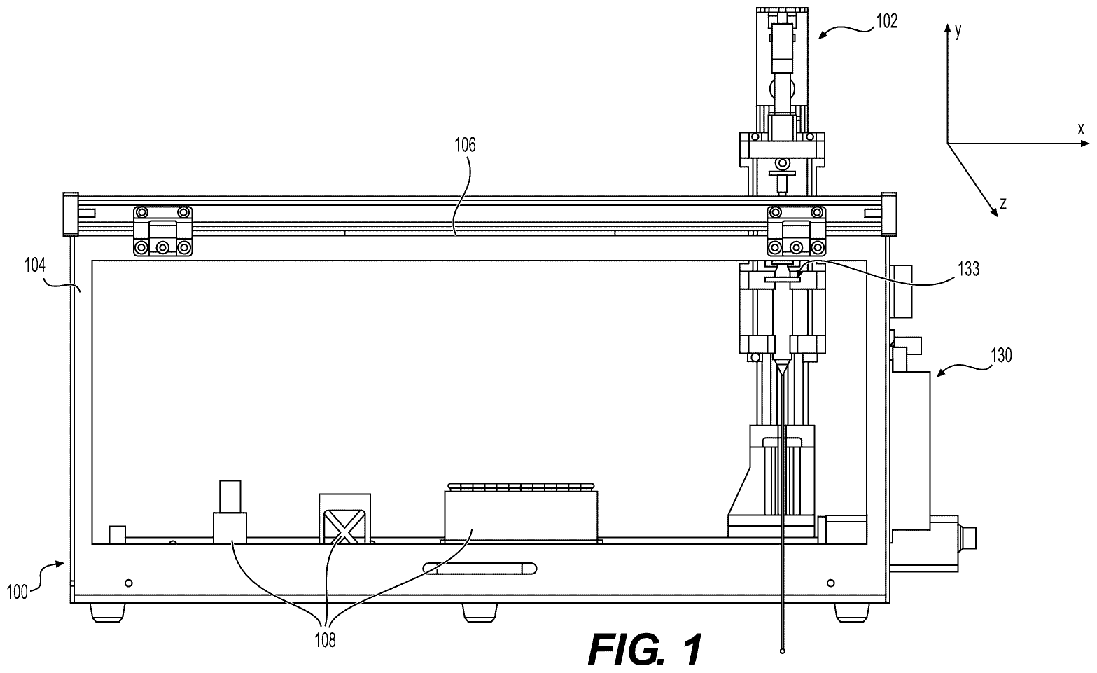

FIGS. 1-4 illustrate an example embodiment of a syringe-based sample collector according to the principles described herein.

The exemplary automated sampling platform 100 employs a disposable syringe/disposable syringes for sample transfer from a sample port to a storage vial. This allows the syringe to be replaced at will (e.g., between each sampling). The system may include a feature to allow for automatically changing out the syringe between samples, which can reduce the possibility of cross-contamination between samples, maintaining the integrity of collected samples.

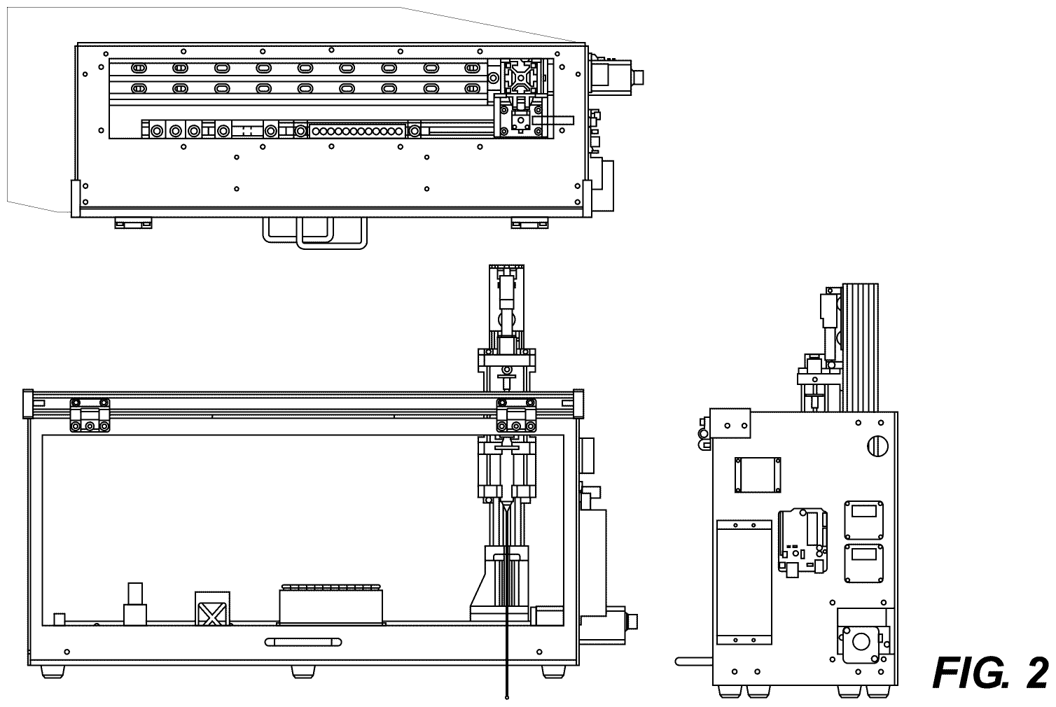

FIG. 1 shows an example automated sampling platform/system 100 in accordance with an illustrative embodiment. In the example shown in FIG. 1, the platform 100 includes a syringe tower 102 that is coupled to an optional workspace enclosure 104. FIG. 2 shows the system 100 of FIG. 1 with the enclosure walls provided. The workspace enclosure may include a UV sterilization lamp 106 and a set of modules 108, such as a sample port, vials, wash, etc.

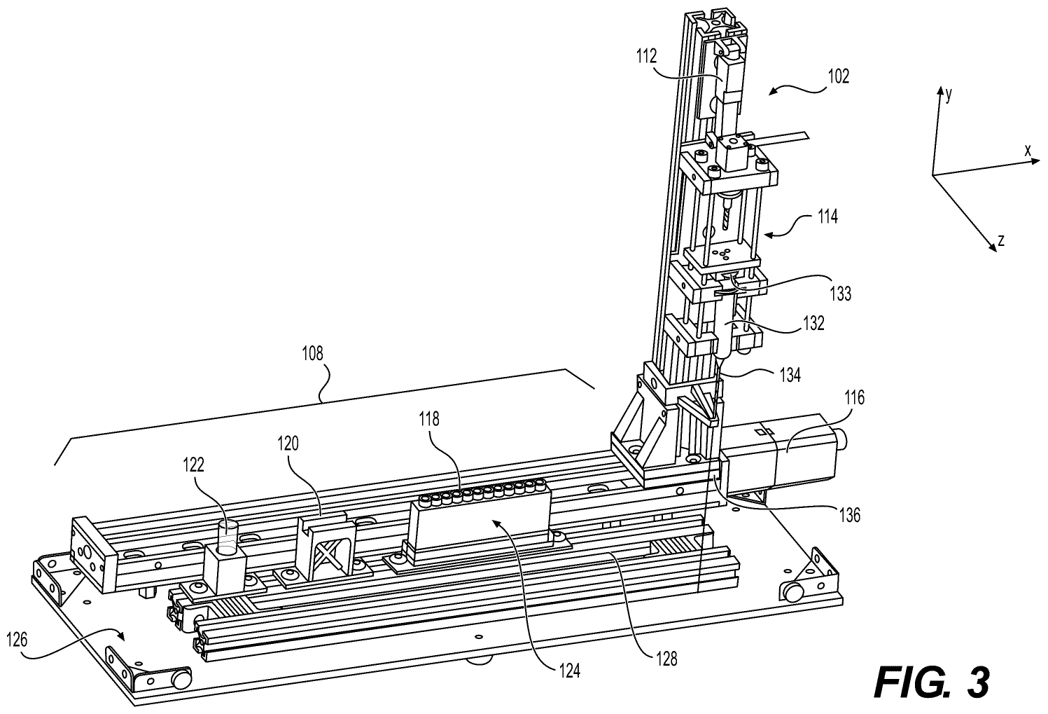

The modules 108 may be removably mounted to a rail 110 (see FIG. 3). The mounting structure of any of the embodiments herein may include a “breadboard” style mounting platform, such as a circuit breadboard or an optical breadboard, such as that shown in https://www.thorlabs.com/newgrouppage9.cfm?objectgroup_ID=159 or the like, although a reconfigurable mounting board is not required. The system includes an electronics panel to provide power to the system and may include a programmable controller, e.g., a microcontroller, or I/O port to be placed in communication with an exterior controller to receive commands as discussed in further detail herein.

FIG. 3 illustrates details of the syringe tower 102 and set of modules 108 of FIG. 1, absent the optional enclosure shown in FIG. 2. The syringe tower 102 includes a vertical actuator 112 and a syringe pump 114. The syringe tower 102 is coupled to a horizontal actuator 116 that is configured to move; in this example, the syringe tower 102 in at least one degree of freedom. In the illustrated embodiment of FIG. 3, various ones of the modules are attached to the system vial a rail 110. In the illustrated embodiment, the rail 10 is an extrusion rail mounted parallel to a movement path of the syringe tower 102 as actuated by the horizontal actuator 116, as described herein. On the rail are mounted modules such as storage vial(s) 118, a sample port mount(s) 120, and a sterilization bath 122, such as an ethanol bath (ethanol in a vial). The storage vials 118 may be in a storage rack 124. In the illustrated embodiment, the various components are mounted on a mounting board 126.

The illustrated horizontal actuator includes a rail or frame 128 on which the syringe tower 102 travels to line up with the various modules 108 on the rail 110 or directly mounted to the mounting board 126.

In various embodiments, it is conceivable that the horizontal actuator 116 or additional actuators (not shown) may be used to move various ones or all of the modules 108, such as a storage vial 118, a sample port mount 120, and an ethanol bath 122 along an extrusion rail 110.

The system includes an electronic panel 130 (FIG. 1) that includes a microcontroller and corresponding electronics to actuate the horizontal actuator 116, syringe pump 114, and vertical actuator 112. The vertical actuator 112 is configured to move the syringe pump/syringe pump assembly 114 to retrieve a disposable syringe (not shown). The disposable syringe may be removably fitted into the syringe pump assembly manually or via an automated system or cartridge, as described herein.

The system 100 is configured to move the syringe pump 114 or and a sample port mount 120 such that the syringe 132 may be aligned with a sample port in the sample port mount 120 so as to engage the sample port to aspirate a sample from the sample port and transfer the sample to a storage vial 118. It is possible for a single disposable syringe 132 to aspirate a sample, transfer it to a vial 118 and then return to the sample port mount 120 to aspirate another sample for transfer to the same or a different vial. In another alternative, the syringe 132 may aspirate enough sample volume to transfer the volume to multiple storage vials 118. Between or after the transfer of the samples, the syringe needle 134, in this example, can be immersed in an ethanol bath 122. Subsequent to the transfer, the disposable syringe 132 may be detached from the syringe pump assembly 114, and a new disposable syringe (not shown) is provided in its place, and the process is repeated for the next sample.

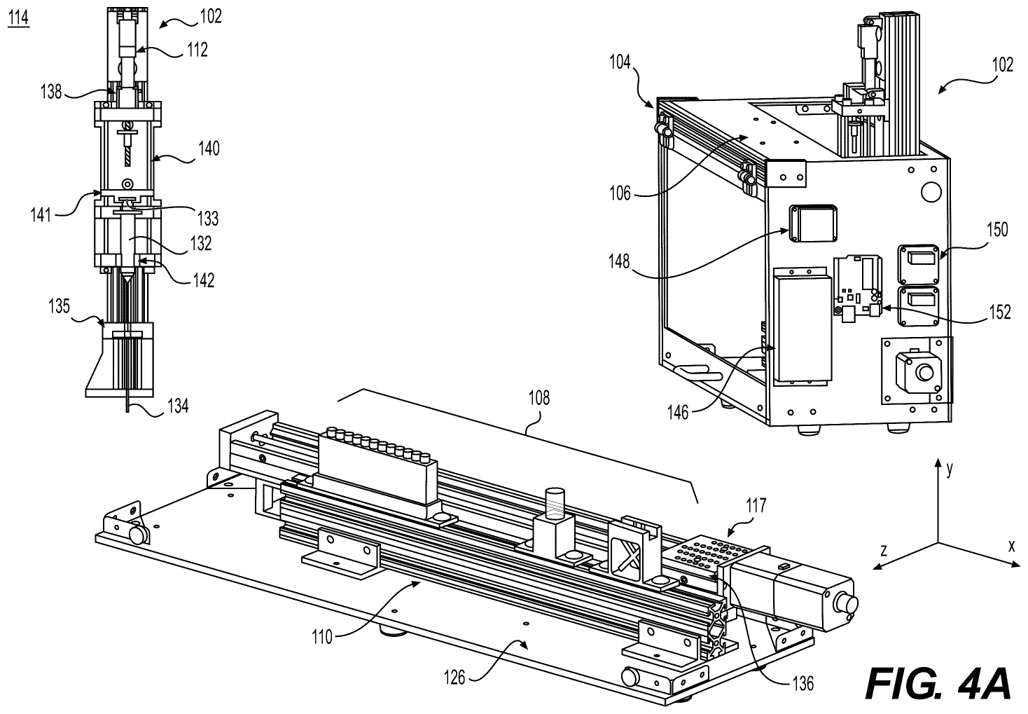

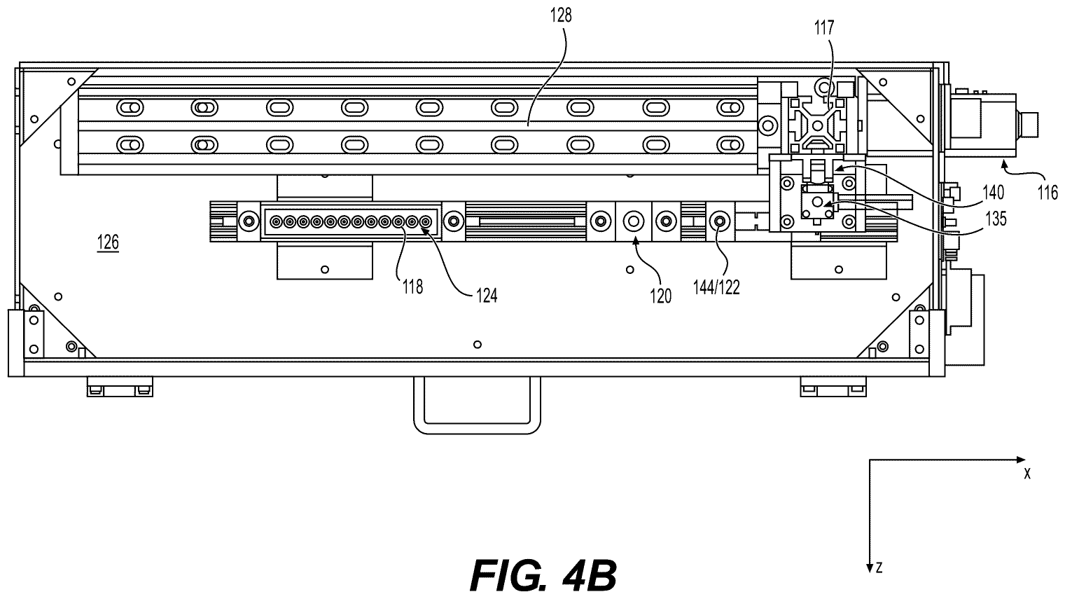

Referring to FIG. 4A, the autosampler 100 may include a custom syringe pump 114 mounted on a 2-degree of freedom gantry mechanism, allowing it to move samples between modules 108 mounted within the device workspace. The horizontal axis actuator 116 with an x-axis linear carriage 117 may be a screw-driven linear carriage that holds the syringe pump subassembly 114 and may be mounted to the device enclosure directly or mounting board 126 and forming the base of the device motion. Mounted on the horizontal carriage 117 may be an extruded rail 110 on which a vertically sliding mounting plate 141 is powered by a rod-style linear servo (a. z-axis actuator) 112. The mounting plate 136 contains the syringe pump 114 itself, a custom assembly that may include a stepper motor 138, lead screw (not shown), and guide rails 140, which actuate the plunger 133 of a connected off-the-shelf syringe via an aspiration carriage 141. The aspiration carriage 141 is a joining piece that couples the stepper motor 138 to the syringe plunger 133, allowing it to aspirate and dispense liquid. The guide rails 140 add rigidity to the assembly as it moves (additional points of contact) and also prevents binding of the actuator (by keeping the plunger inline with the vector of motion). The syringe 132 is fastened into the pump assembly through modular press fit connectors 142, which can be easily replaced to facilitate syringes of different sizes (e.g., a press or snap fit syringe holder). The combination of the above mechanisms leads to a system that can hold a syringe vertically, aspirate and dispense liquid from it, and move it along a vertical planar workspace. The syringe tower 114 can include a needle alignment guide 135 to facilitate the positioning of the needle 134 of the syringe 132 for interaction with the system modules 108.

In keeping with the design philosophy of flexibility, a modular rail 110 or mounting board 126 allows components 108, including two or more sampling ports/port mounts 120 (FIG. 4A(c)), storage vials 118, a syringe storage vial 144 (e.g., ethanol bath 122), and sample trays/rack 124, to be reconfigured depending on the need of a specific application. For example, if multiple points within the bioreactor should be sampled, two sampling ports can be connected to take different types of sample (e.g., one line with cell and another with only cell growth media).

In the present example, the device enclosure 104 may be fabricated from sheet metal, commercial fasteners, 3D printed components, acrylic sheets, and/or may be formed by molding (e.g., injection molded, blow molded, etc.) and/or machined. The device enclosure 104 protects the workspace from outside contaminants and limits exposure of others to the UV disinfecting lamp 106. In an alternative, the system may be completely enclosed, e.g., not having holes present in the illustrated embodiment. Mounted on the underside of the enclosure lid, there may be a sterilizing lamp, e.g., UV-C (b. UV sterilization lamp), which can be used to disinfect the workspace before sampling in case any contaminants are able to enter the system. The system control electronics may be mounted outside the enclosure on the outside (though the placement is not critical for the function of the device). For example, a power supply 146 and relays 148 for actuating the UV lamp 106, motor drivers 150, microcontroller, or the like may be mounted external to the enclosure 104. Various electronics are shown in FIG. 4A, but the electronics are not limited to the illustrated electronics.

The Autosampler is designed to be reconfigurable; behavior changes can be made by modifying the XML behavior file for a sample. As such, the device can be programmed to take samples from different locations, distribute samples across multiple vials, or dilute samples from other reagent vials within the Autosampler. Due to this flexibility, the number and order of steps will vary depending on the experiment or production expansion being executed. The most straightforward configuration of the device involves sampling from a single port into a single vial. In such operation, the Autosampler begins with the needle stored in the ethanol bath when idle to avoid airborne contaminants (Note: The current Autosampler has an open ethanol bath in which the syringe is stored. In a commercialized product, the vial containing the ethanol bath will also have a septum to prevent evaporation of the ethanol). Upon initiation of the sample, the UV lamp turns on, irradiating the workspace of the Autosampler to kill any microorganisms which may have made it inside the device (Note: Disinfecting time can be configured through the user interface). After the lamp has sterilized the area, the vertical axis actuator will lift the syringe out of the ethanol bath and begin sampling.

The horizontal actuator will then move the syringe pump assembly to a prescribed location above an inline sampling septum 154 on the sampling port mount 120, where the needle will then be inserted by the vertical actuator to access the cell media stream. At this point, the syringe pump stepper motor 138 will engage, pulling up on the syringe plunger 133 and aspirating sample volume into the syringe 132. The syringe 132 will then retract, move to a sample vial 118, and insert in the same process as before. Sample vials 118 may be airtight, in which case, efforts should be taken to avoid back-pressure from adding liquid to the vial. To resolve this issue, the syringe plunger 133 may pulse in and out multiple times in order to remove air from the vial 118 as the media is injected. Three cycles of aspirating and dispensing were found to reliably inject the entire collected sample while relieving enough air pressure to avoid stalling the stepper motor controlling dispensing. After the sample has been injected, the syringe 132 retracts and may move back to the ethanol vial 122 and re-insert itself into the sample port 120 (septum 154).

Additional optional steps that may be performed include washing the inside of the syringe with ethanol before sampling, dispensing to a waste vial any remaining residue after injecting the sample, and/or discarding the syringe into a sharps container, as described further herein.

FIGS. 4A and 4B show various components of the automated sampling platform of FIG. 1.

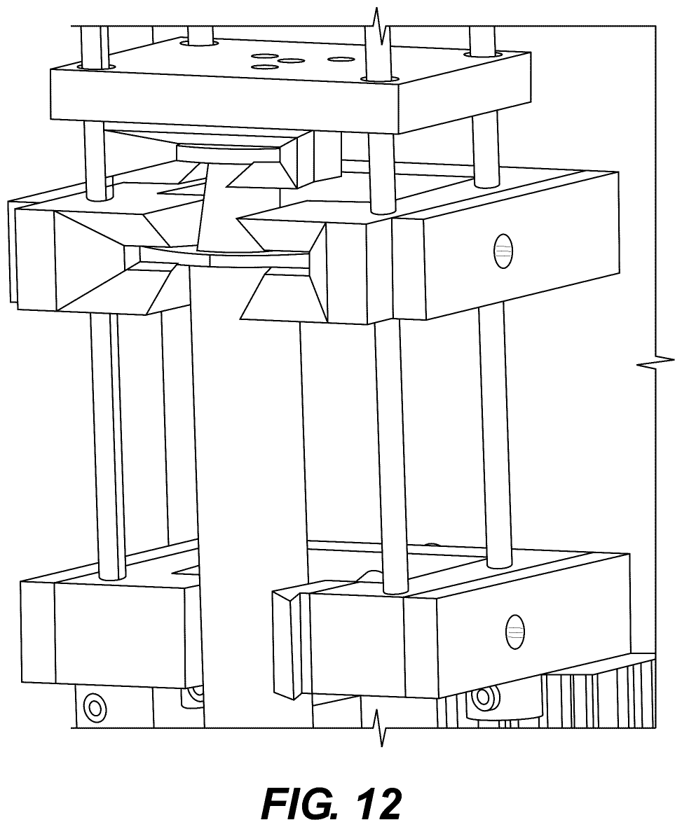

Referring to FIG. 4A, the Autosampler includes a syringe pump that aspirates and dispenses an off-the-shelf needle syringe. The syringe pump subassembly is shown in FIG. 4. Syringes are placed into the aspiration carriage using snap-fit connectors, enabling rapid changeouts. For one example of a syringe holder, see FIG. 12.

In this example, the aspiration carriage is actuated via a stepper motor-driven lead screw. The lead screw and stepper motor were specified to maintain a theoretical step to a millimeter of pump travel of 0.01 mm/step for fine control of withdrawn sample volumes. The syringe pump assembly moves along a vertical segment of extruded framing by actuation of linear servomotor. In this example, the total vertical travel of the syringe may be a range as necessary for the sample size needed, but could be, for example, 30 mm or 50 mm, which is enough to clear both the sampling port and sample storing vials. Of course, the system could be scaled accordingly. To assist with accuracy of needle location, an alignment guide may be provided to surround the needle while it is moving vertically.

The syringe sub-assembly is mounted on a horizontal linear carriage, shown in FIG. 4A(c). The horizontal linear carriage controls the x-axis motion across the workspace. This movement allows the syringe pump to access several modular components mounted on a rail or directly on a mounting board (breadboard). The rail may be a horizontal piece of extruded framing. Each of the sample vials, syringe storage vial, and sampling port mount may be moved on the rail or anywhere on the mounting board. The device may include multiple ones of any of the components. Thus, these modules are configurable, allowing the workspace to adapt to the needs of a specific experiment or manufacturing process. As needed, the modules on the board or rail may include mounts for inline sampling ports, storage vials for the syringe when not in use, and sample vial storage trays. A typical sample consists of the syringe withdrawing from the storage vial, moving to the sampling port to aspirate a sample, and storing in one or more sample vials, before finally returning to the storage vial to await the next sample.

Multiple layers of sterilization and isolation may be utilized to minimize any risk of exposure to the bioreactor and sample. To protect the syringe when not in use, the needle may remain submerged in a vial of ethanol or other suitable disinfectants to disinfect and prevent bacterial contamination from attaching to the needle and contaminating the bioreactor via the sampling port.

Sterilization of the rest of the workspace may be performed with a UV lamp mounted on the underside of the enclosure. Irradiating the workspace before each sample is performed to kill any microorganisms that may have made it into the enclosure. Finally, the enclosure surrounding the device reduces the possible avenues for contamination to enter the system.

The enclosure in the example sub-assembly seen in FIG. 4A is outfitted with the aforementioned UV Sterilization lamp as well as a number of electronics. The electronics are mounted to the side of the enclosure and include an Arduino Uno microcontroller to control motor drivers for the different linear actuators and stepper motors and UV Lamp. The Arduino also communicates with a separate PC running the software suite detailed below. Of course, the microcontroller is not limited to the Arduino but may be any appropriate controller or microcontroller as appropriate for actuating the device as described herein.

In the example device discussed above, the software controlling the Autosampler behavior utilized the Robot Operating System (ROS) framework, a collection of open-source nodes connected by a standardized messaging system. Custom device drivers, behavior control nodes, schedulers, and user interfaces have all been developed to utilize this framework. The example software system is comprised of four main components; the User Interface, Sample Scheduler, Rosplan Executor, and Low-Level Drivers, which are not dependent on any particular operating system. In other words, the systems and methods described herein may be implemented in another framework other than ROS, provided the device functions as described herein.

In the present example, the low-level drivers translate actuator-specific control protocols into the ROS framework, allowing for a common interface to control each of the heterogeneous actuators. Issuing atomic operations may be performed according to ROSPlan, which is a collection of tools for AI logic Planning in a ROS system. The Autosampler may not utilize the planning capabilities of ROSPlan, but instead may simply interface with its plan execution features to allow for sequencing of atomic actions to create a full behavior using human-readable XML files. Researchers without programming experience can modify or recreate behaviors to change how the system operates without needing to recompile any code. Although described with respect to the example as using a ROS framework and ROSPlan functionality, the Autosampler programming may be done in any appropriate framework in any appropriate programming language to provide appropriate functionality according to the principles described herein.

For example, the Autosampler can be programmed to distribute a sample across multiple vials, add stabilizing agents to a sample after it is collected, or even perform non-sampling related behaviors such as dosing reagents from a vial or reservoir into the system via the sampling port.

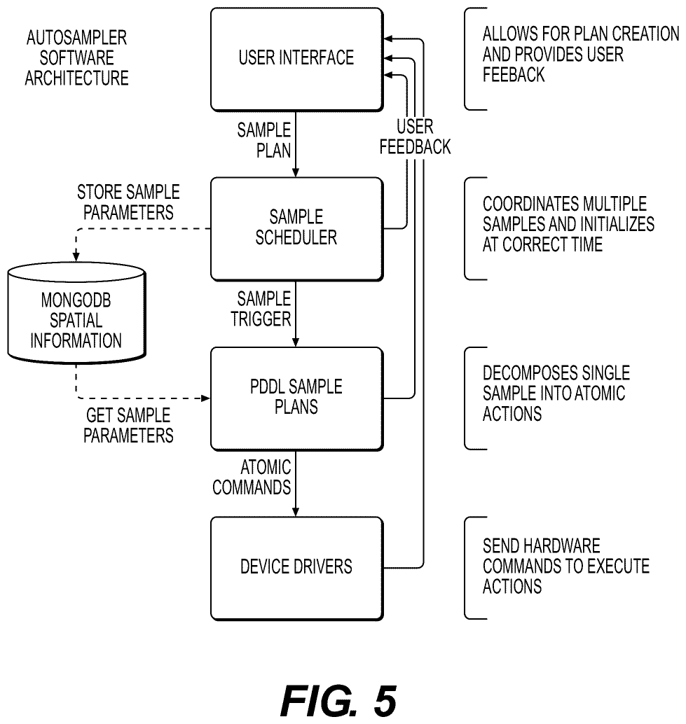

FIG. 5 is a software architecture diagram for software according to the principles described herein. Above the sample plans in the software hierarchy is the sample scheduler, a node that coordinates when individual samples should be executed. The scheduler is implemented as a priority queue to sort samples by execution time along with the necessary parameters for each sample. The scheduler allows multiple sample runs to be completed in parallel in the case of multiple parallel experiments or unique collections for multiple downstream assays. Mirroring the modularity and reconfigurability of the hardware, the software scheduler allows for many different experimental procedures to be conducted automatically without requiring in-depth programming knowledge to modify the machine's behavior.

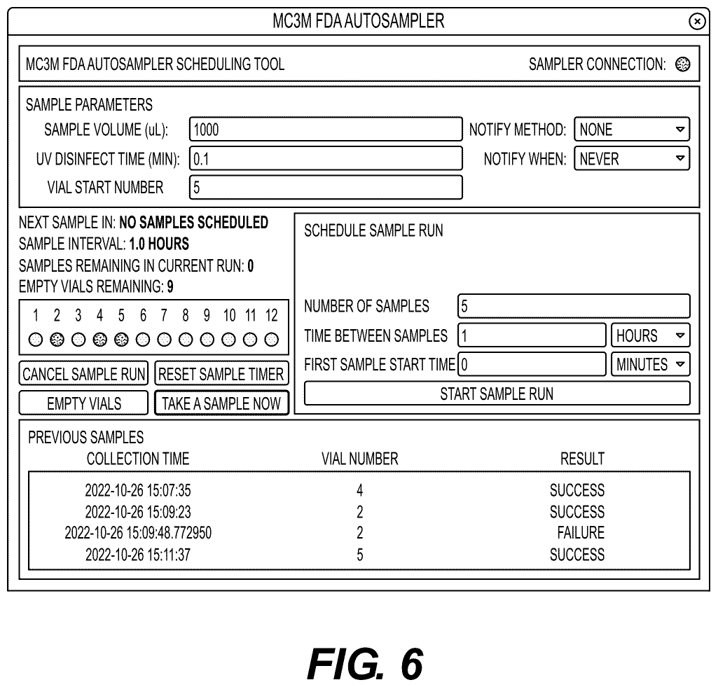

The user interface shown in FIG. 6 provides a graphical dashboard to view the current status of the Autosampler, create and schedule sample runs, and review past samples for success or failure. The dashboard was developed with the PyQT graphics framework and communicated with the autosampler using the roslibpy library, allowing it to be used remotely on a separate PC so that schedules can be created and samples can be reviewed without needing to be physically present in the lab.

Samples were collected from both the Autosampler port and a manual port within the inner recirculation loop throughout the expansion and were then compared to evaluate if any difference or time delay was present due to the placement of the device. No significant difference was found in the metabolites measured between the manual and automated samples.

Contamination of a bioreactor is a tangible and costly threat to both experimental and clinical systems, which can result in loss of reagents and experiment time, and in the clinical setting, can cause delays in treatment, impacting patient health. Any device which interfaces with the bioreactor is a potential source of contamination, and as such, it is important to put as many safeguards in place as possible. During the design of the system, multiple sterilization methods were evaluated in a small survey experiment. During this test, E-Coli bacteria were spiked onto specific places in the bioreactor environment; the sampling port, syringe needle, and vial cap. Multiple sterilization treatments were evaluated for their efficacy in killing these contaminants, including both ethanol and isopropanol sprays (or other suitable disinfectants), as well as UV lamp sterilization. While ethanol is used herein for sterilization of the syringe needle, other sterilization techniques, including isopropanol sprays, without departing from the spirit and scope of the principles described herein.

The implementation described thus far may be modified to provide for the automatic transfer of samples to temperature-controlled storage, as described below. This will prolong the shelf life of samples and enable media with cells to be sampled without requiring immediate intervention from a researcher. Enabling sample freezing can be provided by coupling with a separate automated freezer. The freezer may be positioned such that it can interface with the Autosampler and is controllable from the same ROS framework. Expanding the workspace to include three degrees of motion can enable the system to be utilized with additional storage vehicles, including SBS format well plates and vial racks, or the like. Additionally, an added degree of freedom may eliminate unusable space in front of the workspace rail that may be present in the various configurations of the implementation described above. Increasing the workspace may allow for a greater number of modules to allow for more complicated experiments, such as sampling from multiple independent reactors, interfacing with downstream assay inlets, and increasing sample storage capacity.

In determining the chilling/freezing requirements for samples, a comparative experiment was performed to evaluate protein degradation over 24 hours in a variety of storage conditions. The control group was immediately transferred to a −80 C freezer upon collection, while two other experimental groups were stored at 4 C and room temperature (21 C). After 24 hours, all samples were collected and frozen to −80 C for later evaluation. This procedure aims to mimic the operation of the Autosampler where a collected sample may not be immediately collected. Samples were analyzed. This experiment was not able to determine any significant decay within samples over the time span of 24 hours, though continued experiments are required to determine the full shelf-life of collected samples.

The present implementation of the autosampler includes an integrated biological freezer (−20 C) for sample storage. The previously-described implementation allows for the operator to remove the sample vials manually and insert them into a freezer, and certainly, the present implementation can be used in a similar fashion. The present implementation allows for an operator to perform other tasks instead of needing to stop their task and store the sample or risk damage to it. It was shown that some metabolites and cytokines decay slowly enough for room temperature storage to be sufficient, but for samples including live cells, immediate storage would be beneficial. Thus, while the above-described implementation has utility in some circumstances, the system described below may be useful in other situations.

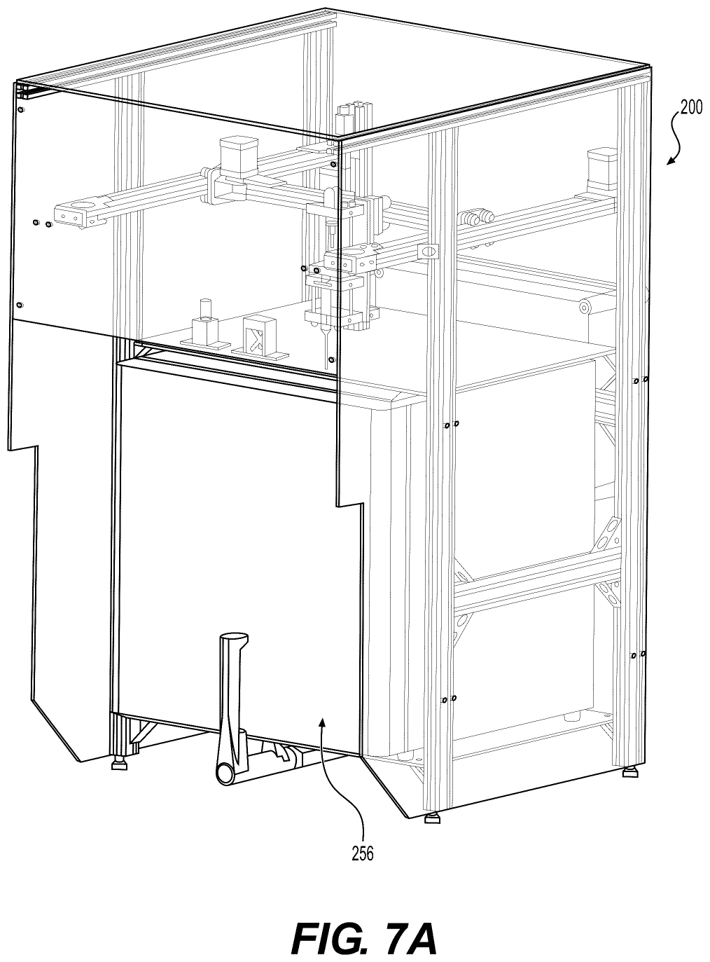

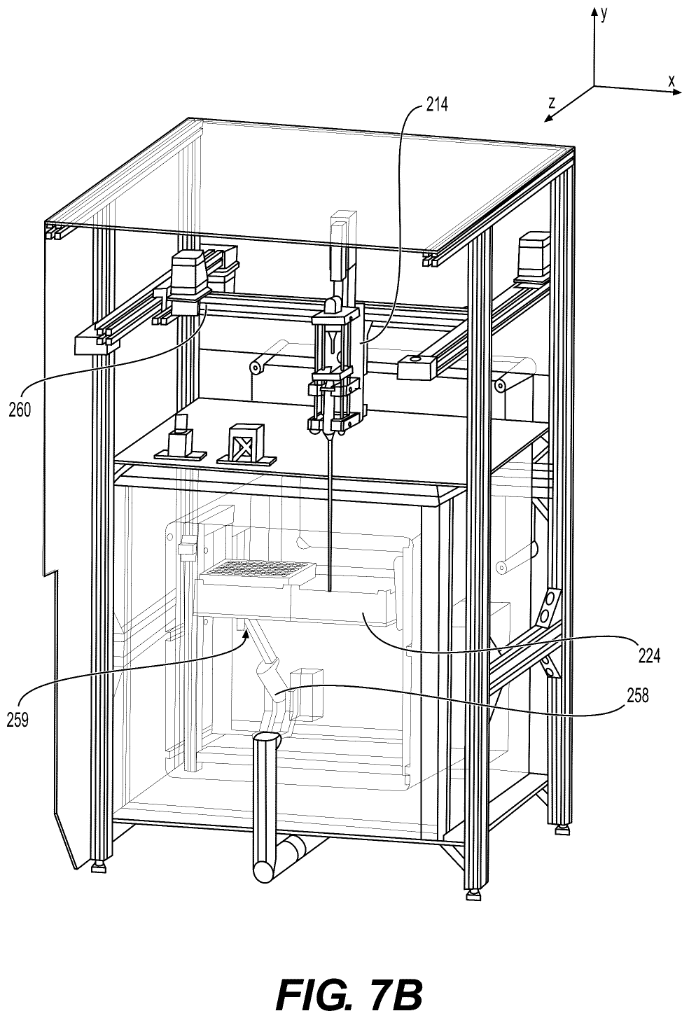

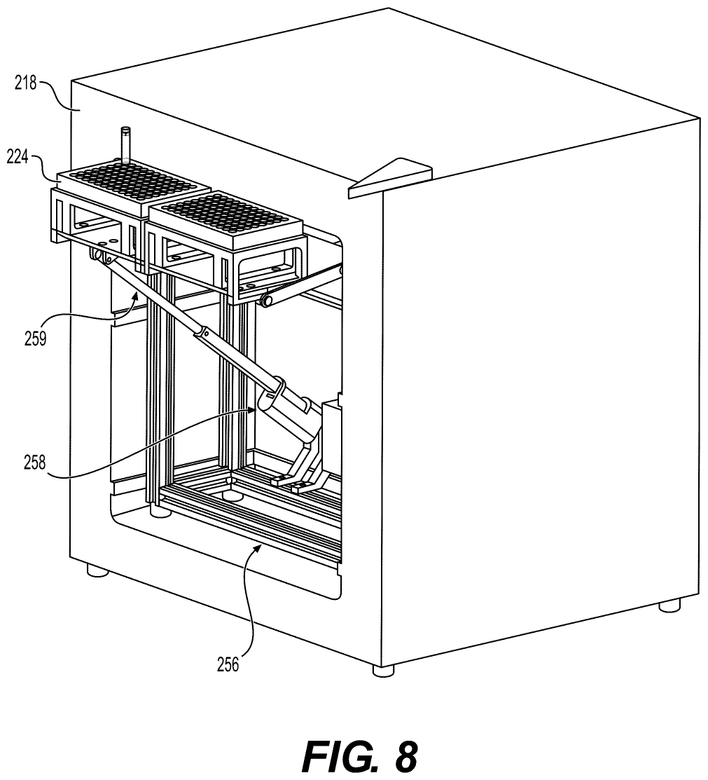

In order to increase the utility and applicability of the autosampler, an automated freezing system was designed. Referring to FIGS. 7A and 7B, an illustrative example system 200 includes a −20 C biological freezer 256. The illustrated design increases the sample storage capacity of 192 samples (two SBS 96 well plate racks) and includes an actuated swinging shelf 261 to move samples 218 out of and into the freezer 256. This illustrative example system 200 enables operator-free sampling and medium-term storage of samples. Referring to FIG. 8, the freeze includes a door actuating linear servo motor 258 and a four-bar linkage 259 to move 2 well plate mounts 224 in and out of the freezer 256.

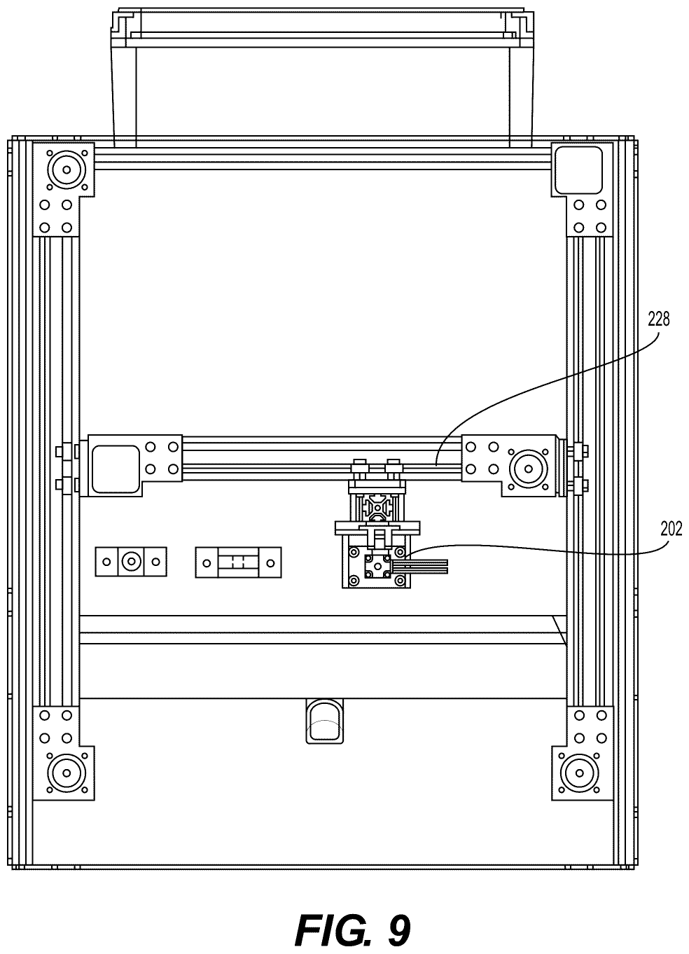

A top surface of the freezer may serve as the workspace for conducting sampling according to the principles described here. FIG. 9 is a top view of the freezer 256 and shows the components of the syringe sampling system that, includes the syringe tower and the rail as described above.

With the addition of the freezer, the 2-degree of freedom actuation previously described may limit the usable area with which to interact with the freezer 256. To fully utilize the device workspace, a new 3 linear axis system 260 is provided. In this illustrative system, there are 2 axes for moving across the entire top of the freezer 256, allowing for a complex arrangement of cleaning and sampling equipment. This design is expandable for future equipment yet to be integrated.

A syringe replacement submodule, which may be used with any example system described herein, may be included within the workspace of the autosampler. The syringe replacement module installs a sterile syringe into the sampler between each sample collection. This will provide additional utility by guaranteeing no cross-contamination between samples. Reuse of a single syringe to retrieve multiple samples may result in a small amount of crossover if the syringe is not completely cleaned between uses. To improve the opportunity for maintaining sterility within the system, a mechanism for using a completely new syringe may be provided.

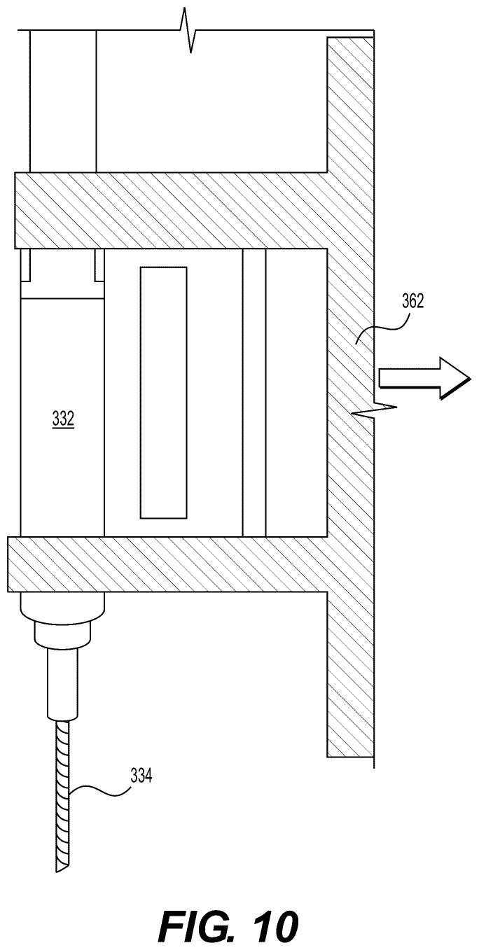

FIG. 10 illustrates an example syringe holder for use according to the principles described herein. While described herein with respect to a workspace on the top surface of the freezer according to the present embodiment, the use of this syringe replacement module is not so limited and may be used in any system described herein or in other systems.

After a sample is taken, the syringe and needle system currently housed in the syringe actuator must be ejected in order to prevent cross-contamination of samples and ensure sterility. Both needles and syringes can be discarded together in a sharps container. For the actuation of this system, the existing 3DoF stage can control the location of the actuator precisely in a 3DoF grid. Within this system, saved cartesian locations can be prescribed for the separate waste containers, which can then be placed next to the fridge assembly. FIG. 10 depicts a concept sketch of a static bar 362 that can be threaded between the press-fit syringe and the syringe actuator. The syringe has 3 points of press-fit contact with the device. From experience with manual syringe exchange, the elements compressing the syringe pump and rim of the syringe body (the upper half of the syringe) are more difficult to align and require more force.

Angling the static bar to apply pressure on the upper half first to disengage may be performed.

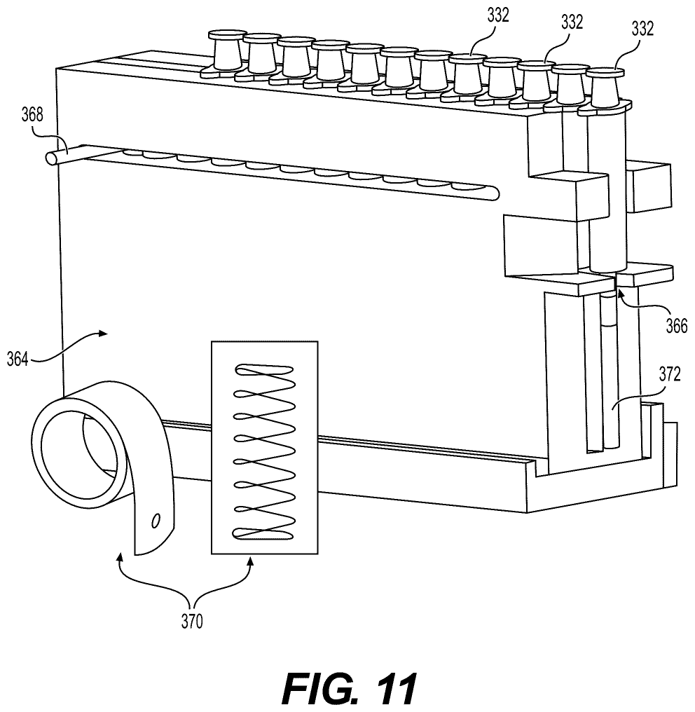

As shown in FIG. 11, the syringe holder system 364 may be designed with the following requirements: a method to stock and hold 12 syringes 332 (to enable up to 12 automatic samples a day), an opening 366 to allow an actuator of the sampling device to grab the syringe 332 in the desired pose, and a re-racking system 368 to line up the next syringe 332 after one is removed. This leads to a spring-loaded syringe cartridge design, as shown in FIG. 11. This system will assume that the syringes 332 will be loaded with the capped and press-fit needles already placed for simplicity since both components come individually sterilized and packed and, therefore, must be racked manually. This entire assembly will also be kept in the autosampler enclosure, which is UV sterilized before every sample.

The holder 364 may be physically designed to provide a surface for the syringe lip and bottom of the syringe to rest on so that they remain upright. The end of the cartridge may be kept open to the size of 1.5 syringe widths and may have a slightly tapered portion sticking out to keep the syringe in place. This design enables an actuator to grasp the syringe in the correct pose to integrate with the syringe actuator (not shown).

The tapered portion may be designed to require less force than the press-fit force of the syringe actuator to enable transfer from one to the other. A spring constant of the spring 370 used to automatically rack new syringes 332 may be designed to provide more force than required to press-fit the syringe into the actuator over a travel of 0.5 syringe widths. These two force profiles can be measured as speed profiles of the stage at first, but as force sensors or limit switches are integrated (as described in the prior section), these force profiles can be directly recorded and controlled to if necessary.

As illustrated in FIG. 11, multiple spring options are also possible for this design. Magazine springs are a good option since they have a large travel while also applying less force the fewer syringes are in the holder. On the one hand, this is a useful function because with fewer syringes, friction, and weight reduce, so less force is required. However, a constant force-coiled spring may be a more robust choice because it would ensure that the same force is applied to each syringe for the press-fit action while also allowing for compliance in the physical design.

This design does not require actuation to re-rack new syringes or interface with the syringe holder for simplicity; however, the syringe replacement system design is not so limited. For example, instead of relying on a precisely designed press-fit holder to keep the syringes in place, a servo-actuated gate can be installed, which opens once the syringe is locked into the holder to release it.

A chained racking system could be used as an alternative, with syringes being spaced out on chain links and a motor progressing the syringes. This way, each link could be designed to perfectly hold the syringe, and both syringe release and re-racking could be accomplished together without a spring by the procession of the motor.

The design of the syringe holder may be varied by the number of syringes that fit in the holder and mechanisms for preventing catching or angling of the stocked syringes, such as varying the wall tolerances and making sure the syringes stay pressed together and flush to the walls and lip they are resting on.

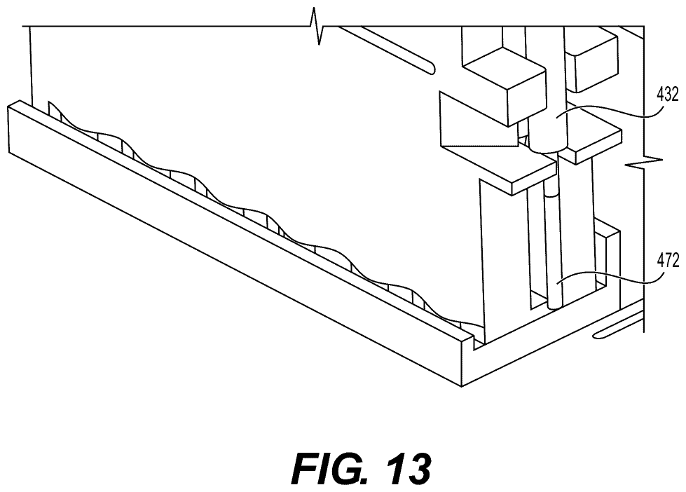

This subsystem may interface with the syringe actuator assembly connected to the linear stage. Regardless of the actuation method, the receiving syringe actuator may include some modifications to allow for inaccurate stage motion and syringe positioning in the holder. For example, in an aspect, slots for the syringe lip and plunger may be exact press-fit sizes and take some manual trial and effort to press-fit. In one design, for example, by introducing sloped slots with a curve on the top and bottom (FIG. 12), inaccuracy in vertical position can be detected from forces on those curved surfaces, with the syringe height being controlled to minimize forces in the upwards or downwards directions. These forces can either be corrected through compliance (FIG. 13) or through speed/force control. Similarly, for example, the horizontal positioning of the syringe body into the snap-fit holder on the syringe actuator may be improved by providing angled rails to lead into the snap-fit and account for the deflection/error of the syringe holder and the stage positioning.

Various aspects of the system described herein may leverage actuation already present in the 3DoF stage to enable the syringe interface without adding additional actuators and sensors. For example, in one aspect, the stage itself may be used to engage with the syringe.

In an aspect, the syringe actuator may be commanded to a setpoint and desired height across from the syringe holder. In this aspect, a speed profile may be used to engage with the syringe as it moves into the syringe holder.

With the addition of force sensors on the syringe actuator body, the force into the syringe can be monitored, and then algorithms for snap-fit can be used to detect a trip point (moment of highest force before the snap-fit engages), after which the force may drop to the spring force of the syringe holder, thereby implying that the syringe was successfully engaged

Feedback controls may be applied to the stage speed to conform to measured desired speed trajectories into the syringe actuator. Additionally, arrays of limit switches or diode receiver arrays can be used to implement bang-bang control to correct positioning errors instead of using force control. The designs may be modified to work with commercially available systems.

The present system may have inherent compliance so that either the syringe actuator or the syringe holder can deflect side to side to account for linear stage positioning precision errors. FIG. 13 shows one particular implementation of mechanical compliance for the syringe holder itself. This solution would allow the syringe holder to move laterally after the syringe makes contact with the angled snap-fit pieces with linear wave springs as the restorative force back to the center.

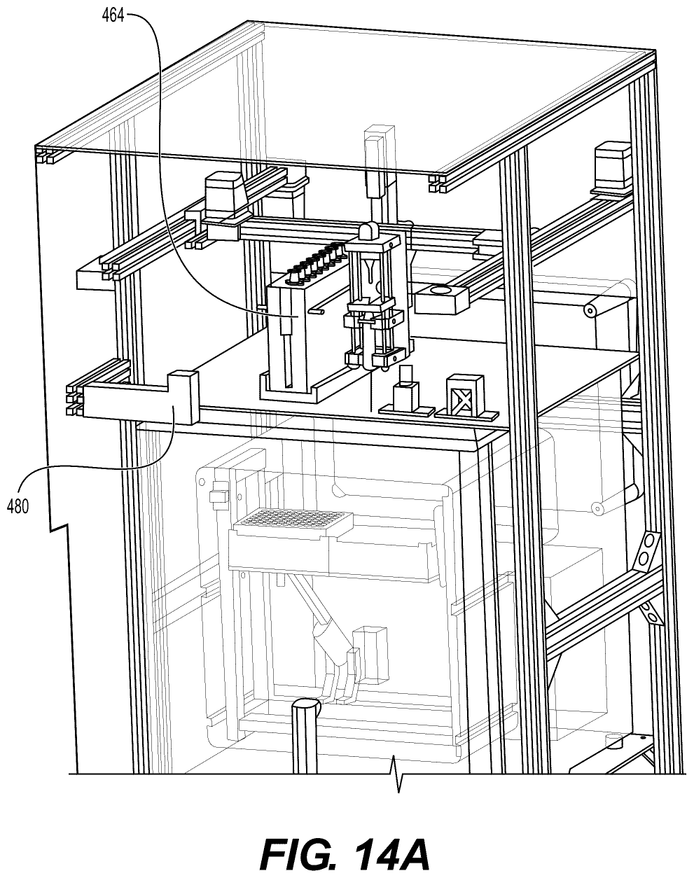

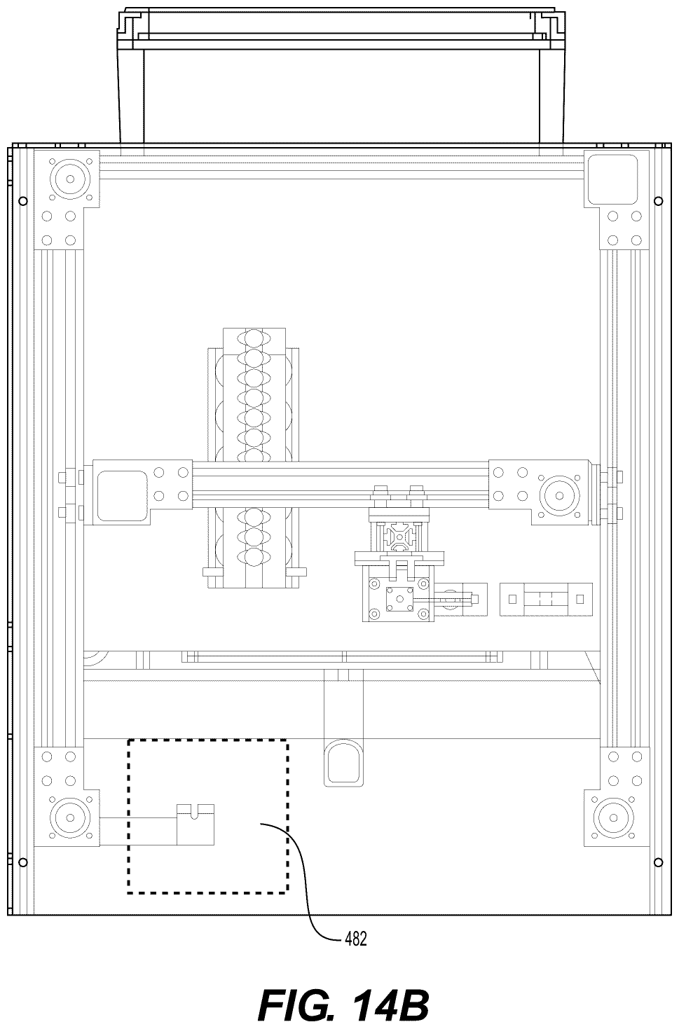

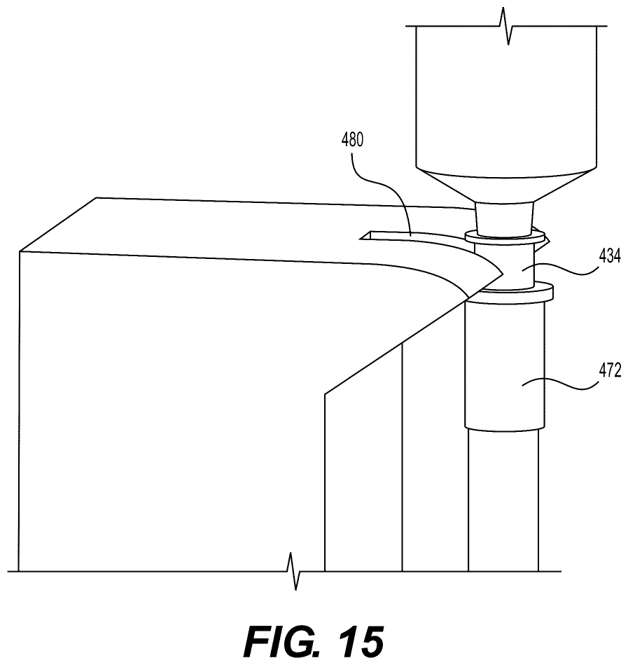

The needle cap 472 is a plastic extruded piece that steps in on the end and hooks onto a lip that encircles the entire needle (FIG. 15). As illustrated in FIGS. 14A, 14B, and 15, the system may include a slot 480 for receiving a portion of the syringe therein and engaging a cap on the needle of the syringe such that movement of the syringe into the syringe pump assembly causes the needle cap to disengage from the syringe. A waste container 482 may be placed below the location of the needle uncapping device to catch the removed needle cap.

Removing this cap without unhooking the needle from the syringe requires large force spikes that would result in oscillations in the syringe actuator block while also creating a projectile out of the syringe cap. In manual operation, it is possible to unhook one edge of the cap by angling the syringe with respect to the needle, after which the cap falls off with no force. Therefore, this unhooking angled force must be applied without an automated system. FIGS. 14A, 14B, and FIG. 15 illustrate one such solution, which involves the 3DoF stage lowering the syringe and needle actuator into an angled slot which gradually applies an angled force on the edge of the cap, therefore unhooking the far edge of the cap. After unhooking, the cap 472 will fall into a designed disposal container below. Success can be determined by including a scale under the waste container which will recognize increments in weight pertaining to the needle cap.

Limit switches can also be triggered by the falling needle cap. Success for this subsystem will be evaluated by visually testing a full cartridge worth of syringes and new needles to confirm that 1) the cap is successfully discarded, 2) the needle tip position relative to the syringe has not changed, and 3) the sensor successfully identifies when needle caps have been discarded.

The example automated sampling platform can operate for any type of sample and collection. With this implemented and verified, the auto-sampler system will be able to take many samples from multiple reactors while ensuring sterility and eliminating cross-contamination between samples, which will relieve manual labor limitations.

While it was originally designed to extract samples from a perfusion bioreactor, such as the FiberCell platform, this automated sampler is able to integrate with any reactor that can be equipped with tubing for circulation or media exchange. The sampler consists of a gantry-mounted syringe that may interface with a rubber septum sampling port. Samples are stored in cryovials which can be directly utilized or frozen down for later analysis. This platform allows for sterilization of its workspace by irradiating the work surface with a UV lamp prior to sampling and additionally by storing the syringe in ethanol when not in use.

In addition to the features described above, the system and software may be configured with different modules or features. For example, the actuator(s) may not have feedback or may have incomplete feedback. For example, a stepper motor for aspirating the syringe may not have feedback, preventing the software from determining if the syringe pump is binding and not aspirating or dispensing the sample correctly. One way to solve this is an encoder that may be added to the syringe pump lead screw to verify position. Additionally, the z-axis actuator may be replaced with a position-controlled version, allowing the device to detect failures in puncturing the vial septum or failures caused by x-positioning errors, among other advantages.

A needle-based automated sampler, according to principles described herein, provides an effective method to sample from a line of tubing without required excess dead volume. It also eliminates the need for the reuse of tubing, minimizing the risk of cross-contamination of samples. This platform solves a problem in the space of cell manufacturing research, which currently requires inconvenient and potentially contaminating manual sample collections. It accomplishes this while eliminating complicated tubing systems which require excess waste media to be thrown out, as well as taking steps to minimize any possibility of contamination. An automated sampling system may include a syringe sampling assembly; and a sampling platform comprising: a mounting structure; at least one sample storage rack and at least one corresponding sample container; a movable carriage assembly fixably coupled to the mounting structure, the movable carriage assembly comprising a movable carriage configured to carry at least one syringe sampling assembly; a sampling port mount fixably coupled to, or relative to, the mounting structure, the sampling port mount having one or more samples of interest (static or flowing); the syringe sampling assembly coupled to the mounting structure, the syringe sampling assembly having one or more actuated assemblies configured, and directed via a controller, to (i) attachably engage a syringe from a set of syringes, (ii) position the syringe at the sampling port mount, (iii) actuate the syringe to draw a sample from the one or more samples of interest, (iv) position the syringe at the corresponding sample container, (v) actuate the syringe to expel a portion of the sample into the corresponding sample container, (vi) disengage the syringe, and (vii) attachably engage another syringe from a set of syringes for drawing a another sample from the one or more samples of interest different from the previously drawn sample.

An automated sampling system may include a sampling platform comprising a mounting structure; a movable carriage; a sampling port mount; and a sample storage rack; and a syringe pump assembly removably coupled to the movable carriage.

The mounting structure may comprise a mounting board. The movable carriage is movable in a first direction (e.g., an +/−x direction) in a plane parallel to the mounting board.

The sample storage rack may be fixedly connected to the mounting structure.

The at least one sample storage rack may be movably connected to the mounting structure.

The sampling platform may further include a guide rail along which the movable carriage moves.

The system may further include a motor coupled to the movable carriage to cause movement of the carriage in at least one direction.

The motor may be a stepper motor or wherein the motor is coupled to a driven lead screw to cause the movement of the movable carriage.

The sampling platform may include a syringe storage configured to receive the syringe after being disengaged.

The syringe may include a needle for engaging the sample port.

The system may further comprise needle storage, such as a storage vial.

The system may include a disinfectant in the needle storage (e.g., ethanol).

The system may further include a motor assembly (e.g., comprising a z-axis motor, e.g., stepper motor) coupled to the movable vertical carriage to cause movement of the syringe carriage (e.g., along the Z axis) (e.g., wherein the syringe pump assembly includes a driven lead screw coupled to the syringe motor to cause the movement of the syringe carriage).

The syringe sampling assembly may include a syringe holder configured to attachably engage the syringe; a syringe carriage (e.g., movable vertical carriage) coupled to the syringe holder for moving the syringe holder toward and away from the sampling platform; and a syringe actuator (e.g., z-axis actuator) positioned to actuate a plunger of a removable syringe in the syringe holder.

The syringe actuator may be capable of actuating a plunger of the syringe in a second direction (e.g., +Z and −Z direction).

The syringe actuator may include an aspiration motor, wherein the aspiration motor is a stepper motor, or wherein the aspiration motor is coupled to a driven lead screw to actuate the plunger of the removable syringe.

The syringe actuator may include an aspiration carriage configured to actuate the plunger of the syringe at a predetermined amount per actuation (e.g., 0.01 mm/step).

The syringe holder may be a snap-fit syringe holder.

The syringe holder may include a cantilever lug that is bendable to insert the removable syringe and returns to an unstressed position after insertion of the syringe to secure the removable syringe.

The syringe sampling assembly may include a needle alignment guide.

The system may include a device enclosure enclosing at least a portion of the sampling platform and at least a portion of the syringe sampling assembly.

The system may include a disinfecting lamp located in the device enclosure (e.g., a UV sterilization lamp).

The sampling port mount may comprise a rubber septum placed in-line within a tubing-based system housing the one or more samples of interest.

The sample storage rack may be movable with respect to the mounting board.

The system may include a storage unit for storing the sample storage rack.

The storage unit may be a temperature-controlled unit (e.g., capable of reaching refrigerating or freezing temperatures for storing samples contained in the sample storage rack) (e.g., comprising a cryo-vial and, in an aspect, a sealable cryo-vial).

The system may include a gantry (e.g., movable in at least four degrees of freedom; or less) coupled to the sample storage rack configured to move the sample storage rack to and/or from the sampling platform to a storage unit.

The gantry may be configured to exchange the sample storage rack for an additional sample storage rack in the storage unit and provide the additional sample storage rack to the sampling platform.

The sample storage may be configured to hold a line or array of sample containers (e.g., vials) (e.g., n×m matrix of vials).

The system may include a processor; and a memory having instructions stored thereon, wherein execution of the instructions by the processor, causes the processor to cause the system to perform at least one of any of a plurality of steps described herein.

The system may further include a user interface (e.g., a graphic user interface).

The system may further include the set of syringes in corresponding syringe holders.

Each syringe holder may correspond to a separate syringe pump assembly.

The system may include a corresponding syringe storage vial for each syringe.

The system may include a syringe cartridge coupled to a syringe pump assembly such that a syringe in the syringe cartridge is injected into the syringe pump assembly by a spring force provided by the syringe cartridge. The syringe storage cartridge may be a stand-alone system capable of being used with systems other than the system described herein.

The system may include a slot for receiving a portion of the syringe therein and engaging a cap on the needle of the syringe such that movement of the syringe into the syringe pump assembly causes the needle cap to disengage from the syringe.

The slot may be angled to allow for mechanical compliance.

A method of autosampling biosamples includes using the automated sampling system may include moving the syringe via the moveable carriage to the sampling port mount, inserting a needle of the syringe into a sampling port held in place by the sampling port mount, aspirating a sample, extracting the needle from the sampling port, moving the syringe via the moveable carriage to a vial in the sample storage rack, ejecting the sample into the vial, and returning the needle to the sampling port or to a storage vial.

The method may further include withdrawing the syringe from the storage vial before moving the syringe to the sampling port mount.

Vertical movement of the syringe and actuation of the syringe may be provided by the syringe pump assembly.

The method may further include moving the vial to a storage unit after at least one sample has been ejected into the vial.

The method is used to operate the system as described herein.

A non-transitory computer-readable medium may be provided having instructions stored thereon, wherein execution of the instructions by the processor causes the processor to perform any one of the methods described herein.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

It must also be noted that, as used in the specification and the appended claims, the singular forms “a,” “an,” and “the” include plural referents unless the context clearly dictates otherwise. Ranges may be expressed herein as from “about” or “5 approximately” one particular value and/or to “about” or “approximately” another particular value. When such a range is expressed, other exemplary embodiments include from the one particular value and/or to the other particular value.

By “comprising” or “containing” or “including” is meant that at least the name compound, element, particle, or method step is present in the composition or article or method, but does not exclude the presence of other compounds, materials, particles, method steps, even if the other such compounds, material, particles, method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

As discussed herein, a “subject” may be any applicable human, animal, or other organism, living or dead, or other biological or molecular structure or chemical environment, and may relate to particular components of the subject, for instance, specific tissues or fluids of a subject (e.g., human tissue in a particular area of the body of a living subject), which may be in a particular location of the subject, referred to herein as an “area of interest” or a “region of interest.”

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, 4.24, and 5).

Similarly, numerical ranges recited herein by endpoints include subranges subsumed within that range (e.g., 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

The construction and arrangement of the systems and methods as shown in the various exemplary embodiments are illustrative only. Although only a few embodiments have been described in detail in this disclosure, many modifications are possible (e.g., variations in sizes, dimensions, structures, shapes and proportions of the various elements, values of parameters, mounting arrangements, use of materials, colors, orientations, etc.). For example, the position of elements may be reversed or otherwise varied, and the nature or number of discrete elements or positions may be altered or varied. Accordingly, all such modifications are intended to be included within the scope of the present disclosure. The order or sequence of any process or method steps may be varied or re-sequenced according to alternative embodiments. Other substitutions, modifications, changes, and omissions may be made in the design, operating conditions, and arrangement of the exemplary embodiments without departing from the scope of the present disclosure.

Some references, which may include various patents, patent applications, and publications, are cited in a reference list and discussed in the disclosure provided herein. The citation and/or discussion of such references is provided merely to clarify the description of the present disclosure and is not an admission that any such reference is “prior art” to any aspects of the present disclosure described herein. In terms of notation, “[n]” corresponds to the n^{th} reference in the list. All references cited and discussed in this specification are incorporated herein by reference in their entireties and to the same extent as if each reference was individually incorporated by reference.

Examples of samples and collection operations that may be implemented by the exemplary automated sampling platform or systems that can implement the automated sampling operation described herein are described in [1]-[18], each of which is incorporated by reference herein in its entirety.

The following patents, applications and publications as listed below and throughout this document are hereby incorporated by reference in their entirety herein.
[1] Matheus C. Carvalho, Rachel H. Murray, “Osmar, the open-source microsyringe autosampler,” HARDWARE ARTICLE, VOLUME 3, P10-38, Apr. 1, 2018 (https://doi.org/10.1016/j.ohx.2021.e00177).
[2] Haby B, Hans S, Anane E, et al., Integrated Robotic Mini Bioreactor Platform for Automated, Parallel Microbial Cultivation With Online Data Handling and Process Control. SLAS TECHNOLOGY: Translating Life Sciences Innovation. 2019;24(6):569-582. (doi:10.1177/2472630319860775https://doi.org/10.1177/24726303198607 75).
[3] CTCanalytics, CTC A200S Autosampler Operating Manual-Microsoft Word—CTC A200S
[4] Autosampler Data Sheet.doc (ietltd.com)
[5] Longwell S, Fordyce P, “micrIO: an open-source autosampler and fraction collector for automated microfluidic input-output,”
[6] C. Barckhausen, B. Rice, S. Baila, L. Sensebé, H. Schrezenmeier, P. Nold, H. Hackstein, and M. T. Rojewski, “GMP-Compliant Expansion of Clinical-Grade Human Mesenchymal Stromal/Stem Cells Using a Closed Hollow Fiber Bioreactor,” Methods in Molecular Biology (Clifton, N.J.), vol. 1416, pp. 389-412, 2016.
[7] A. L. Russell, R. C. Lefavor, and A. C. Zubair, “Characterization and cost-benefit analysis of automated bioreactor-expanded mesenchymal stem cells for clinical applications,” Transfusion, vol. 58, pp. 2374-2382 October 2018.
[8] A. Mizukami and K. Swiech, “Mesenchymal stromal cells: From discovery to manufacturing and commercialization,” Stem Cells Int., vol. 2018, p. 4083921, April 2018.
[9] “Aseptic, reusable sampling system for bioreactors.” https://www.inforsht.com/en/bioreactors/bioreactor-accessories/supersafesampler/.
[10] “Sample pilot sp100.” http://mastsampling.com/products/sample-pilot/.
[11] “Seg-flow s3.” https://optimalbiotech.com/product/automated-samplingsystems/.
[12] C. Cannizzaro and U. V. Stockar, “Device for automated bioreactor sampling,” Jun. 2007.
[13] B. Haby, S. Hans, E. Anane, A. Sawatzki, N. Krausch, P. Neubauer, and M. N. C. Bournazou, “Integrated robotic mini bioreactor platform for automated, parallel microbial cultivation with online data handling and process control,” SLAS TECHNOLOGY: Translating Life Sciences Innovation, vol. 24, no. 6, pp. 569-582, 2019. PMID: 31288593.
[14] P. Rohe, D. Venkanna, B. Kleine, R. Freudl, and M. Oldiges, “An automated workflow for enhancing microbial bioprocess optimization on a novel microbioreactor platform,” Microbial Cell Factories, vol. 11, p. 144, October 2012.
[15] J. P. Efromson, S. Li, and M. D. Lynch, “Biosamplr: An open source, low cost automated sampling system for bioreactors,” HardwareX, vol. 9, p. e00177, 2021.
[16] A. Hofer, P. Kroll, M. Barmettler, and C. Herwig, “A reliable automated sampling system for on-line and real-time monitoring of cho cultures,” Processes, vol. 8, no. 6, 2020.
[17] A. Hajila, G. Muller, and S. Olivier, “Self-sealing septum device,” Sep. 2013.
[18] L. Rónai and T. Szabo, “Snap-fit assembly process with industrial robot including force feedback,” Robotica, vol. 38, pp. 1-20, 05 2019.f8

While various embodiments of the present invention have been described above, it should be understood that they have been presented by way of example only, and not limitation. It will be apparent to persons skilled in the relevant art that various changes in form and detail can be made therein without departing from the spirit and scope of the present invention. Thus, the breadth and scope of the present invention should not be limited by any of the above-described exemplary embodiments, but should be defined only in accordance with the following claims and their equivalents.

1. An automated sampling system comprising:
a syringe sampling assembly; and
a sampling platform comprising:
a mounting structure;
at least one sample storage rack and at least one corresponding sample container;
a movable carriage assembly fixably coupled to the mounting structure, the movable carriage assembly comprising a movable carriage configured to carry at least one syringe sampling assembly;
a sampling port mount fixably coupled to, or relative to, the mounting structure, the sampling port mount having one or more samples of interest;
the syringe sampling assembly coupled to the mounting structure, the syringe sampling assembly having one or more actuated assemblies configured, and directed via a controller, to (i) attachably engage a syringe from a set of syringes, (ii) position the syringe at the sampling port mount, (iii) actuate the syringe to draw a sample from the one or more samples of interest, (iv) position the syringe at the corresponding sample container, (v) actuate the syringe to expel a portion of the sample into the corresponding sample container, (vi) disengage the syringe, and (vii) attachably engage another syringe from a set of syringes for drawing another sample from the one or more samples of interest different from a previously drawn sample.

2. (canceled)

3. The automated sampling system of claim 1, wherein the mounting structure comprises a mounting board, and wherein the movable carriage is movable in first direction in a plane parallel to the mounting board.

4-6. (canceled)

7. The automated sampling system of claim 1, wherein the sampling platform further comprises a guide rail along which the movable carriage moves, the sampling system further comprising a motor or motor assembly coupled to the movable carriage to cause movement of the movable carriage along at least one direction.

8. (canceled)

9. The automated sampling platform of claim 7, wherein the motor or motor assembly is a stepper motor or wherein the motor or motor assembly is coupled to a driven lead screw to cause the movement of the movable carriage.

10. The automated sampling system of claim 1, the sampling platform further comprising a syringe storage configured to receive the syringe after being disengaged, wherein the syringe comprises a needle for engaging a sample port.

11-14. (canceled)

15. The automated sampling system of claim 1, wherein the syringe sampling assembly comprises:
a syringe holder configured to attachably engage the syringe;
a syringe carriage coupled to the syringe holder for moving the syringe holder toward and away from the sampling platform; and
a syringe actuator positioned to actuate a plunger of a removable syringe in the syringe holder.

16. (canceled)

17. The automated sampling system of claim 15, wherein the syringe actuator comprises an aspiration motor, wherein the aspiration motor is a stepper motor, or wherein the aspiration motor is coupled to a driven lead screw to actuate the plunger of the removable syringe.

18. The automated sampling system of claim 15, wherein the syringe actuator comprises an aspiration carriage configured to actuate the plunger of the syringe at a predetermined amount per actuation.

19. The automated sampling system of claim 15, wherein the syringe holder is a snap-fit syringe holder.

20. The automated sampling system of claim 15, wherein the syringe holder comprises a cantilever lug that is bendable to insert the removable syringe and returns to an unstressed position after insertion of the syringe to secure the removable syringe.

21-23. (canceled)

24. The automated sampling system of claim 1, wherein the sampling port mount comprises a rubber septum placed in-line within a tubing-based system housing the one or more samples of interest.

25-27. (canceled)

28. The automated sampling system of claim 1, further comprising a gantry coupled to the at least one sample storage rack configured to move the at least one sample storage rack to and/or from the sampling platform to a storage unit.

29. The automated sampling system of claim 28, wherein the gantry is configured to exchange the at least one sample storage rack for an additional sample storage rack in the storage unit and provide the additional sample storage rack to the sampling platform.

30. The automated sampling system of claim 1, wherein the sample storage rack is configured to hold a line or array of sample containers.

31-35. (canceled)

36. The automated sampling system of claim 1, further comprising a syringe cartridge coupled to a syringe pump assembly such that a syringe in the syringe cartridge is injected into the syringe pump assembly by a spring force provided by the syringe cartridge.

37. The automated sampling system of claim 36, further comprising a slot for receiving a portion of the syringe therein and engaging a cap on a needle of the syringe such that movement of the syringe into the syringe pump assembly causes the needle cap to disengage from the syringe, wherein the slot is angled to allow for mechanical compliance.

38. (canceled)

39. A method of autosampling biosamples, comprising:
using an automated sampling system, the automated sampling system comprising:
a syringe sampling assembly; and
a sampling platform comprising:
a mounting structure;
at least one sample storage rack and at least one corresponding sample container;
a movable carriage assembly fixably coupled to the mounting structure, the movable carriage assembly comprising a movable carriage configured to carry at least one syringe sampling assembly;
a sampling port mount fixably coupled to, or relative to, the mounting structure, the sampling port mount having one or more samples of interest;
the syringe sampling assembly coupled to the mounting structure, the syringe sampling assembly having one or more actuated assemblies configured, and directed via a controller, to implement the method comprising:
moving a syringe from a set of syringes via the movable carriage assembly to the sampling port mount,
inserting a needle of the syringe into a sampling port held in place by the sampling port mount,
aspirating a sample,
extracting the needle from the sampling port,
moving the syringe via the movable carriage to a vial in the sample storage rack,
ejecting the sample into the vial, and
returning the needle to the sampling port or to a storage vial.

40. The method of claim 39, further comprising withdrawing the syringe from the storage vial before moving the syringe to the sampling port mount.

41. The method of claim 39, whereby vertical movement of the syringe and actuation of the syringe are provided by a syringe pump assembly.

42. The method of claim 39, further comprising moving the vial to a storage unit after at least one sample has been ejected into the vial.

43-44. (canceled)
